(19) United States
(12) Patent Application Publication
(10) Pub. No.: US 2020/0114450 A1
(43) Pub. Date: Apr. 16, 2020

(54) Augmented Reality in a Material Processing System

(71) Applicant: Hypertherm, Inc., Hanover, NH (US)

(72) Inventors: Dennis Kulakowski, Corinth, VT (US); Christopher S. Passage, Sunapee, NH (US); Richard Adams, Norwich, VT (US); Clifford G. Darrow, Lyme, NH (US); Brett A. Hansen, Mapleton, UT (US); John Peters, Canaan, NH (US); Justin Gullotta, Brownsville, VT (US); Garrett K. Quillia, Enfield, NH (US); Jeff Ortakales, Newbury, NH (US)

(21) Appl. No.: 16/654,412

(22) Filed: Oct. 16, 2019

Related U.S. Application Data
(60) Provisional application No. 62/746,176, filed on Oct. 16, 2018.

(51) Int. Cl. B23K 9/095; B23K 10/00; B23K 9/32; G09B 19/24; A61F 9/06

(52) U.S. Cl. CPC B23K 9/0956; B23K 10/006; A61F 9/06; B23K 9/322; G09B 19/24; B23K 9/0953

(57) ABSTRACT

A method for visually communicating material processing parameters to an operator of a torch system. The method includes receiving data related to a material processing operation from at least one sensor of a torch system and at least one camera disposed on a protective helmet. The method further includes processing the data into information relating to a set of material processing parameters and converting the information into visual data compatible with a display disposed on or within the protective helmet. The method also includes providing the visual data to a region of the display for viewing by an operator of the torch system. The region of the display is within a field of view of the operator in order to visually communicate the information to the operator of the torch system.

### CROSS REFERENCE TO RELATED APPLICATION

This application claims the benefit of and priority to U.S. Provisional Patent Application No. 62/746,176, filed Oct. 16, 2018, the entire contents of which are owned by the assignee of the instant application and incorporated herein by reference in their entirety.

### FIELD OF THE INVENTION

The present invention relates generally to material processing systems, including systems and methods for providing information to operators of material processing systems using augmented reality.

### BACKGROUND OF THE INVENTION

Operators and technicians of material processing systems have limited information readily available to them while operating with these systems. For example, plasma cutting systems frequently display the set amperage and processing settings on the system itself rather than on the torch or anywhere proximate where the actual process is being performed and the operator is located. Further, operators and technicians are not supplied with real time feedback, relying instead on trial and error or acquired skill to tune a system or perform an operation properly (e.g., only inspecting a cut after it has been performed). For example, in plasma cutting, there is typically no feedback to the power supply to adjust the actual performance of the arc relative to the workpiece.

The current method of vision during a plasma cutting operation (even with auto-tint) allows the user to only see a small halo of visibility around the cutting arc and work through limited visibility, sounds, and feel to know where and how to cut. There is also a lot of system feedback that can only by noticed after a cut by looking at the power supply for amperage, fault codes, etc. Additionally, when servicing or repairing these systems, technicians are often required to look back and forth between a manual and the system itself to identify relevant parts and proper techniques, and/or describe to a remote technician over the phone what they are looking at and dealing with. This results in inefficient and lengthy repair and maintenance times (e.g., prolonged down times).

Therefore, there is a need to create a system which receives and/or captures a set of system and environment inputs, analyzes these inputs, and displays this analysis in real time information to an operator or technician. This would create a feedback loop so that the system and/or operator can dynamically adapt to situations to improve material processing quality (e.g., cut quality) and maintenance procedures and performance.

### SUMMARY OF THE INVENTION

Accordingly, an object of the invention is to provide information related to a material processing operation to an operator of a torch system. It is an object of the invention to provide information related to a material processing operation to an operator of a torch system wearing a protective helmet. It is an object of the invention to provide information related to a material processing operation to an operator of a torch system using an augmented reality system. It is an object of the invention to capture information related to a material processing operation and adjust material processing parameters based on the captured information.

In some aspects, a method for visually communicating material processing parameters to an operator of a torch system includes receiving, from at least one sensor of a torch system, first data related to a material processing system. The method further includes receiving, from at least one camera disposed on a protective helmet, second data related to the material processing operation. The method also includes processing the first and second data into information relating to a set of material processing parameters. The method further includes converting the information into visual data compatible with a display disposed on or within the protective helmet. The method also includes providing the visual data to a region of the display for viewing by an operator of the torch system. The region of the display being within a field of view of the operator.

In some embodiments, the torch system includes a torch and a workpiece. In some embodiments, the at least one sensor is disposed on or within the torch. For example, the at least one sensor can include at least one of an accelerometer or a gyroscope. In some embodiments, the at least one sensor is configured to monitor motion of the torch during the material processing operation. In some embodiments, the set of material processing parameters includes at least one of a velocity of the torch with respect to the workpiece and an angle of the torch with respect to the workpiece.

In some embodiments, the method further includes receiving, from at least one temperature sensor disposed on or within the protective helmet, third data related to the material processing operation. For example, the method can include processing the third data into temperature information relating to a temperature of a region of the workpiece. In some embodiments, the method further includes converting the temperature information into second visual data compatible with the display and providing the second visual data to the region of the display for viewing by the operator of the torch system. In other embodiments, the second visual data includes an alert indicating the temperature of the region of the workpiece.

In some embodiments, the method further includes receiving, from a light spectrometer disposed on or within the protective helmet, third data related to the material processing operation. For example, the method can include processing the third data into wavelength information relating to a wavelength of a light emitted from the torch system. In some embodiments, the method further includes converting the wavelength information into second visual data compatible with the display and providing the second visual data to the region of the display for viewing by the operator of the torch system.

In some embodiments, the method further includes receiving, from a microphone disposed on or within the protective helmet, audio data related to a command from the operator of the torch system. For example, the method can include processing the visual data into adjusted visual data based on the command from the operator of the torch system. In some embodiments, the method further includes providing the visual data to the region of the display for viewing by the operator of the torch system.

In other embodiments, the method further includes transferring the visual data to a second display located at a distance from the protective helmet. In some embodiments, the method also includes providing the visual data to a second region of the second display for viewing by a second operator.

In some aspects, a method for visually communicating material processing parameters to an operator of a torch system includes receiving, from at least one camera disposed on a protective helmet, first data related to a material processing operation of a torch system. The torch system includes a torch and a workpiece. The method further includes receiving, from the at least one camera disposed on the protective helmet, second data related to a set of fiducials disposed on a surface of the workpiece. The set of fiducials are shaped to visually convey a reference scale. The method also includes processing the second data into reference information relating to the reference scale and processing, using the reference information, the first data into information relating to a set of material processing parameters. The method further includes converting the information into visual data compatible with a display disposed on or within the protective helmet and providing the visual data to a region of the display for viewing by an operator of the torch system. The region of the display being within a field of view of the operator.

In some embodiments, the set of fiducials are equally spaced apart. In some embodiments, the set of fiducials includes at least two anchor fiducials. In some embodiments, the set of processing parameters includes at least one of a velocity of the torch with respect to the workpiece and an angle of the torch with respect to the workpiece.

In some embodiments, the method further includes receiving, from at least one temperature sensor disposed on or within the protective helmet, third data related to the material processing operation. For example, the method can include processing the third data into temperature information relating to a temperature of a region of the workpiece. In some embodiments, the method further includes converting the temperature information into second visual data compatible with the display and providing the second visual data to the region of the display for viewing by the operator of the torch system. In some embodiments, the second visual data includes an alert indicating the temperature of the region of the workpiece.

In some embodiments, the method further includes receiving, from a light spectrometer disposed on or within the protective helmet, third data related to the material processing operation. For example, the method can include processing the third data into wavelength information relating to a wavelength of a light emitted from the torch system. In some embodiments, the method further includes converting the wavelength information into second visual data compatible with the display and providing the second visual data to the region of the display for viewing by the operator of the torch system.

In some embodiments, the method further includes receiving, from a microphone disposed on or within the protective helmet, audio data related to a command from the operator of the torch system. For example, the method can include processing the visual data into adjusted visual data based on the command from the operator of the torch system. In some embodiments, the method further includes providing the visual data to the region of the display for viewing by the operator of the torch system.

In other embodiments, the method further includes transferring the visual data to a second display located at a distance from the protective helmet. In some embodiments, the method also includes providing the visual data to a second region of the second display for viewing by a second operator.

In some aspects, a method for controlling material processing parameters of a torch system includes receiving, from a torch system including a torch and a workpiece, first data related to a set of desired material processing parameters for a material processing operation of the torch system. The method further includes receiving, from at least one camera disposed on a protective helmet, second data related to the material processing operation of the torch system. The method also includes processing the second data into information relating to a set of material processing parameters and calculating, based on the information, at least one of the set of material processing parameters. The method further includes determining, based on the first data, at least one of the set of desired material processing parameters. The method also includes comparing the at least one of the set of material processing parameters and the at least one of the set of desired material processing parameters and, in response to the comparing, transferring, to the torch system, a set of adjusted material processing parameters.

In some embodiments, the at least one of the set of material processing parameters includes a velocity of the torch relative to the workpiece and the at least one of the set of desired material processing parameters includes a desired velocity of the torch relative to the workpiece. For example, determining that the velocity of the torch is different than the desired velocity of the torch can result in the transferring of the set of adjusted material processing parameters. In some embodiments, one of the set of adjusted material processing parameters includes an operating current of the torch.

In some embodiments, the at least one of the set of material processing parameters includes a length of the material processing operation and the at least one of the set of desired material processing parameters includes a desired length of the material processing operation. For example, determining that the length is greater than or equal to the desired length can result in the transferring of the set of adjusted material processing parameters. In some embodiments, the method further includes ceasing the material processing operation of the torch system.

In some embodiments, the at least one of the set of material processing parameters includes a distance between the torch and an edge of the workpiece and the at least one of the set of desired material processing parameters includes a threshold distance between the torch and the edge of the workpiece. For example, determining that the distance between the torch and the edge of the workpiece is less than or equal to the threshold distance can result in the transferring of the set of adjusted material processing parameters. In some embodiments, the method further includes initiating a torch shutdown sequence at the torch system.

Other aspects and advantages of the invention can become apparent from the following drawings and description, all of which illustrate the principles of the invention, by way of example only.

### BRIEF DESCRIPTION OF THE DRAWINGS

The advantages of the invention described above, together with further advantages, may be better understood by referring to the following description taken in conjunction with the accompanying drawings. The drawings are not necessarily to scale, emphasis instead generally being placed upon illustrating the principles of the invention.

FIG. 1 is an isometric view of an exemplary protective helmet including an augmented reality system, according to an embodiment of the invention.

FIG. 2 is a block diagram of an exemplary system including the protective helmet shown in FIG. 1 and an exemplary torch system, according to an embodiment of the invention.

FIG. 3 is an illustrative representation of an exemplary display of the protective helmet shown in FIG. 1, according to an embodiment of the invention.

FIG. 4 is an illustrative representation of an exemplary display of the protective helmet shown in FIG. 1, according to an embodiment of the invention.

FIG. 5 is an illustrative representation of an exemplary display of the protective helmet shown in FIG. 1, according to an embodiment of the invention.

FIG. 6 is a flow diagram of method steps for visually communicating material processing parameters to an operator of the torch system shown in FIG. 2, according to an embodiment of the invention.

FIG. 7 is a flow diagram of method steps for visually communicating material processing parameters to an operator of the torch system shown in FIG. 2, according to an embodiment of the invention.

FIG. 8 is a flow diagram of method steps for controlling material processing parameters of the torch system shown in FIG. 2, according to an embodiment of the invention.

### DETAILED DESCRIPTION OF THE INVENTION

In some aspects, the systems and methods described herein can include one or more mechanisms or methods for providing information related to a material processing operation to an operator of a torch system. The system and methods can include one or more mechanisms or methods for providing information related to a material processing operation to an operator of a torch system wearing a protective helmet. The systems and methods described herein can permit an operator of a torch system to receive information related to a material processing operation using an augmented reality system. The system and methods described herein allow for a torch system to adjust material processing parameters based on captured information related to a material processing operation.

In some aspects, the systems and methods described herein identify information that can be presented to a wearer of an augmented reality system (e.g., welding goggles, a welding helmet, smart glasses, etc.). In some embodiments the augmented reality system creates an augmented reality experience via a set of system, workpiece, and environmental inputs which are processed by the augmented reality system to create and relay the desired data. The use of an augmented reality system mitigates the above described problems (e.g., incorrect processes, poor cut quality, inefficient operation, low visibility, improper and/or inefficient maintenance procedures, etc.) by providing an operator or technician real time system data and instruction in an easily understandable format which is overlaid on the components at issue during the process/procedure.

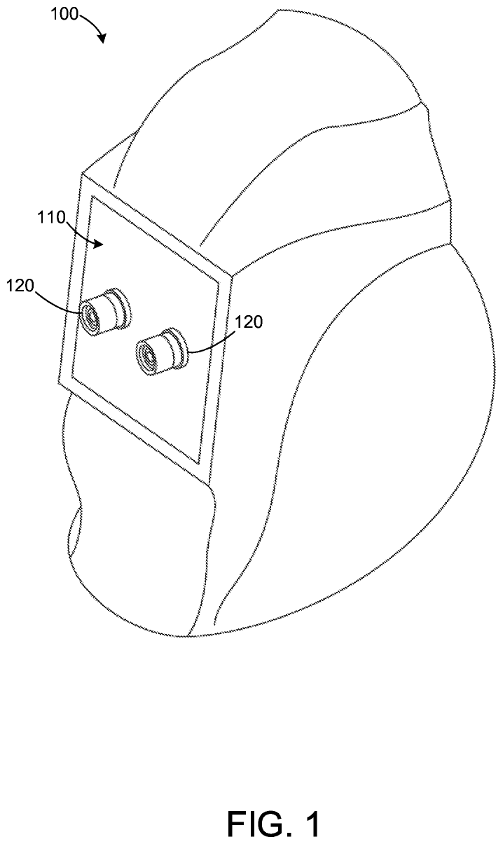

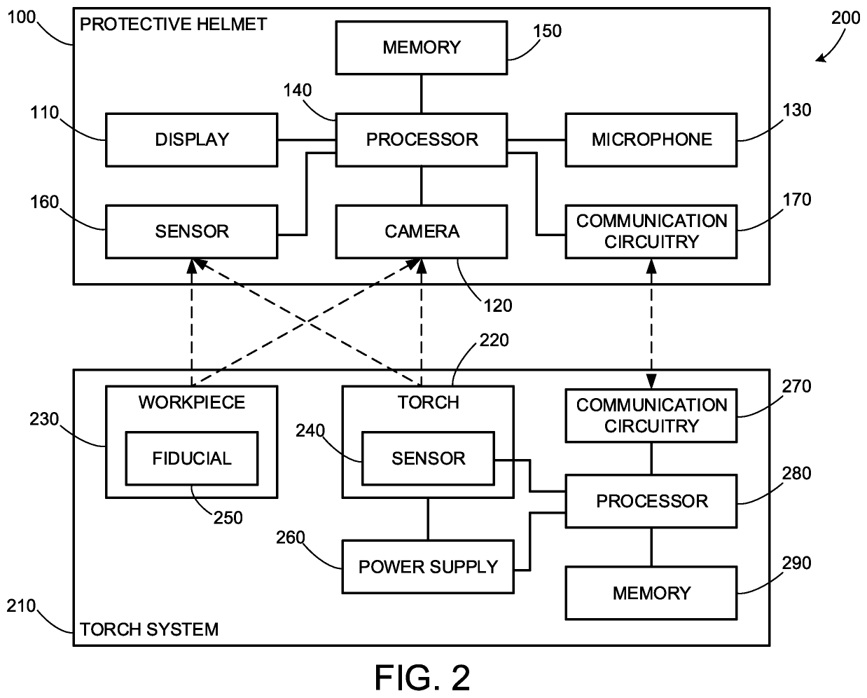

In one aspect, a device (e.g., an augmented reality system) incorporated into a protective helmet provides real time optical feedback and virtual overlay onto an operator's field of vision, thereby providing several critical pieces of information to improve the operator's vision. An augmented reality system combines captured video and generated graphics to produce an image integrated with reality giving the impression that the operator's vision is enhanced. The operator experiences this augmented reality through display devices located within the operator's field of vision. For example, the augmented reality system can provide an operator with awareness of system status, awareness of work piece geography relative to a torch, and assistance with component identification for maintenance and repair procedures. Referring to FIGS. 1-2, an augmented reality system 200 includes a protective helmet 100 and a torch system 210. The protective helmet 100 includes input devices that are configured to receive data corresponding to a material processing operation. For example, in some embodiments protective helmet 100 includes at least one camera 120, a microphone 130, and at least one sensor 160. The protective helmet 100 also includes output devices that are configured to provide data to the operator and the torch system 210. For example, in some embodiments protective helmet 100 includes a display 110 and communication circuitry 170. The protective helmet 100 also includes processor 140 and memory 150 to process the data received by the input devices and process the data that will be delivered by the output devices.

The torch system 210 includes a workpiece 230 and a torch 220 that is configured to cut the workpiece 230. The torch 220 is powered by a current and a voltage delivered by a power supply 260. In some embodiments, the torch system 210 also includes processor 280, memory 290, and communication circuitry 270. In some embodiments, communication circuitry 270 of the torch system 210 is communicatively coupled to the communication circuitry 170 of the protective helmet 100 in order to transfer data between the protective helmet 100 and the torch system 210. Communication circuitry 170 and communication circuitry 270 can use Bluetooth, Wi-Fi, or any comparable data transfer connection. In some embodiments, torch 220 includes at least one sensor 240 that is configured to collect data corresponding to the material processing operation. For example, in some embodiments the sensor 240 is disposed on or within torch 220. In some embodiments, sensor 240 can include at least one of an accelerometer or a gyroscope that can be configured to sense if and how the torch 220 is positioned and/or moving. For example, an accelerometer could indicate if the torch 220 is moving at a constant speed, accelerating, or decelerating.

The input devices of the protective helmet 100 can function individually or together to receive data corresponding to the material processing operation. For example, the camera 120 of the protective helmet 100 can be configured to take images and live video of the workpiece 230. In some embodiments, the camera 120 can be a high-resolution camera that is capable of determining tolerances and other similar characteristics of the workpiece 230. In some embodiments, the camera 120 is configured to capture high dynamic range (HDR) video in order to visualize the torch 220 and workpiece 230 with a higher dynamic range. HDR live video allows an operator to see the torch system 210 with greater clarity and depth. In some embodiments, the camera 120 is a smartphone connected to the protective helmet 100 and configured to function as both camera 120 and display 110. In some embodiments, the protective helmet 100 includes two cameras 120, each configured to capture video corresponding to one of the operator's two eyes. The augmented reality system 200 can process the captured video from the two cameras 120 using processor 140 to generate a 3D video. The generated 3D video can be displayed to the operator using display 110. For example, one-half of display 110 can be dedicated to display a portion of the 3D video configured for one of the eyes of the operator while the other half of display 110 can be dedicated to display another portion of the 3D video configured for the other eye of the operator.

The protective helmet 100 allows an operator to see the workpiece 230 clearly without the tint or dimness of traditional eye protection. The protective helmet 100 can include one or more sensors 160 that can receive data corresponding to the material processing operation. For example, the sensor 160 can include an infrared or temperature sensor which can be used to target the workpiece 230 and let the operator know the temperature of the workpiece 230 in order to avoid burns to the operator and detect if there is too large of a heat affected zone on the workpiece 230 being cut. The system 200 can adjust when the workpiece 230 hits certain heat thresholds. For example, if a workpiece is getting too hot, the system 200 can pause during the cut so as not to overheat and/or warp the piece. In some embodiments, sensor 160 is an infrared sensor which can identify pierce puddles.

In one embodiment, the sensor 160 can include an RFID sensor to identify the type of consumables or other system components with an RFID tag. In one embodiment, the system 200 can use RFID data to determine the remaining consumable life of a system component. In one embodiment, the RFID scanner can be used to identify the type of consumables in the torch 220 and notify the operator if there is a mismatch between the selected currents and type of consumables loaded. In some embodiments, the sensor 160 can include a light spectrometer to measure the wavelength or color of the light captured by the sensor 160. This information can be used to give the operator feedback about cutting conditions, such as cut speed. Color could also be used to identify potentially hazardous materials in a weld being gouged based upon the color of the light of the burning material.

The protective helmet 100 can include a microphone 130 which can receive audio commands from the operator, allowing for hands-free control of the system 200. For example, the operator can issue a command to the microphone 130 to overlay a shape or pattern on the workpiece 230 using display 110. In some embodiments, the microphone 130 can be used to receive audio data corresponding to the material processing operation. For example, in plasma cutting, there is a notable audio change when a plate pierce is complete. The microphone 130 can receive this sound as an audio input and inform the operator when the pierce has been completed. In some embodiments, audio commands can be used to signal the completion of a job or work order.

In some embodiments, the workpiece 230 and/or torch 220 includes one or more fiducials 250 that are disposed on the surface of the workpiece 230 and/or torch 220 and are shaped to visually convey a reference scale. The fiducials 250 can include scales or other known shapes and objects that are attached to the workpiece 230 so as to provide a frame of reference or scale for analysis software. In one embodiment, the fiducials 250 can convey information corresponding to the locations of sensors 240 on or in the torch 220 relative to one another and the operators. In one embodiment, the fiducials 250 include a Torch Anchor Point. The torch anchor point could include at least one scale or known sized piece to enable accurate visual analysis of other features relative to the known size or reference. In one embodiment the one or more fiducials 250 can be generated by/projected from the torch 220 as a set of laser points and/or shapes projected from a known location on torch 220 onto the workpiece 230. With known locations and angles at the torch 220 the size and spacing of the laser images on workpiece 230 can be used by the processor 280 to analyze torch position and/or plasma processes.

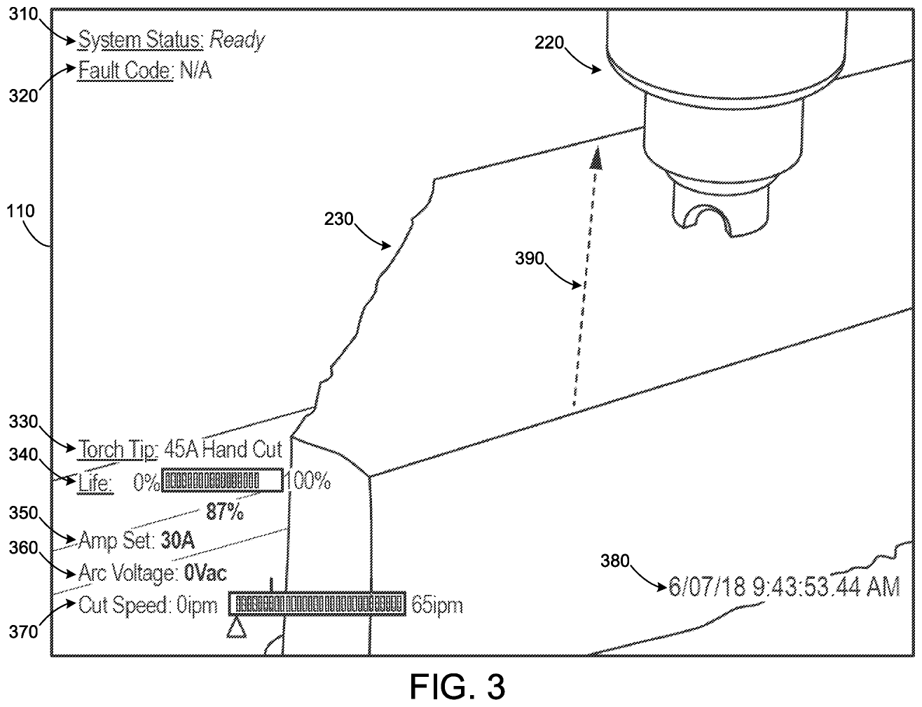

FIGS. 3-5 show an exemplary plasma cutting operation as viewed through a display 110 disposed in a protective helmet 100 of augmented reality system 200. It is understood that this is just an example of the capabilities of the augmented reality system 200 and that its uses can be applied to many material processing operations such as waterjet and laser, among others. Further, applicability of augmented reality system 200 extends beyond material processing operations to also maintenance and repair of material processing systems themselves.

Referring to FIG. 3, an example display 110 of the protective helmet 100 shows an exemplary precut display within the protective helmet 100 as it would be seen by an operator of the torch system 210, according to embodiments of the invention. In FIG. 3, an operator has recently performed a plasma cutting operation and is about to perform another plasma cutting operation, as is readily ascertainable from the display 110 where a number of visual data elements are overlaid onto the operator's field of view. The visual data elements include system status 310, fault code indicator 320, torch process type 330, torch tip life indicator 340, amperage setting indicator 350, arc voltage indicator 360, cut speed indicator 370, and date and time indicator 380. The display also shows the torch 220 and the workpiece 230. The operator can quickly see that the torch system 210 is ready, there are no fault codes, what the consumable life status is, what process it is set up to perform, the date and time, and the ideal cut speed for the current settings. Further a proposed or desired cut path with workpiece angularity 390 is overlaid on the workpiece 230 to direct the motion of the torch 220 during the cutting operation (e.g., allowing an operator to trace along a known/easily visible line with torch 220 to achieve a desired result). The display can also show a nest of desired parts for the workpiece 230, a grid overlaid on the workpiece 230, and/or an entire desired cut pattern.

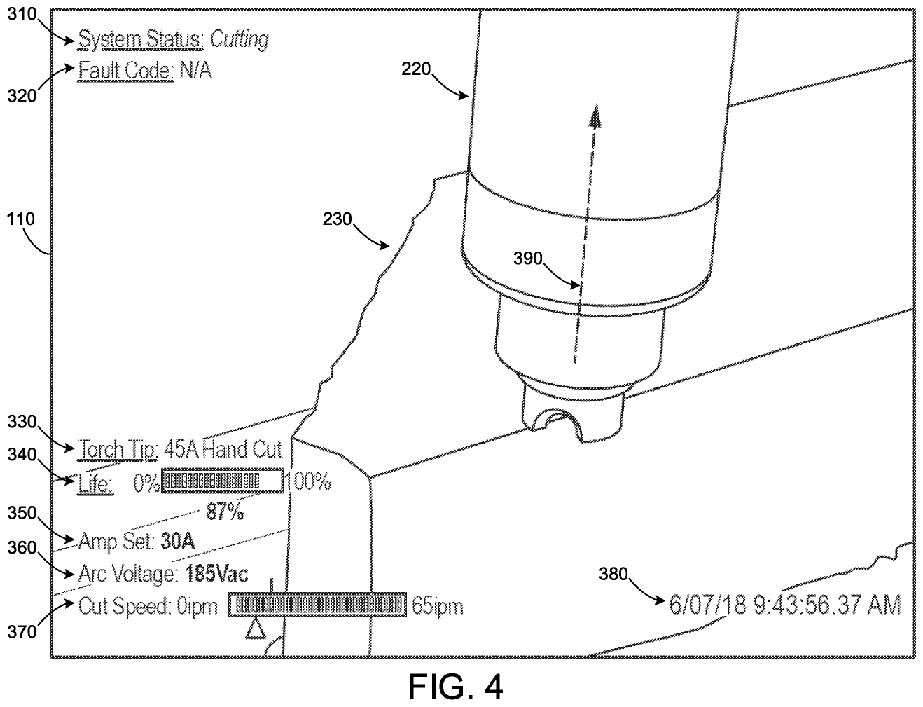

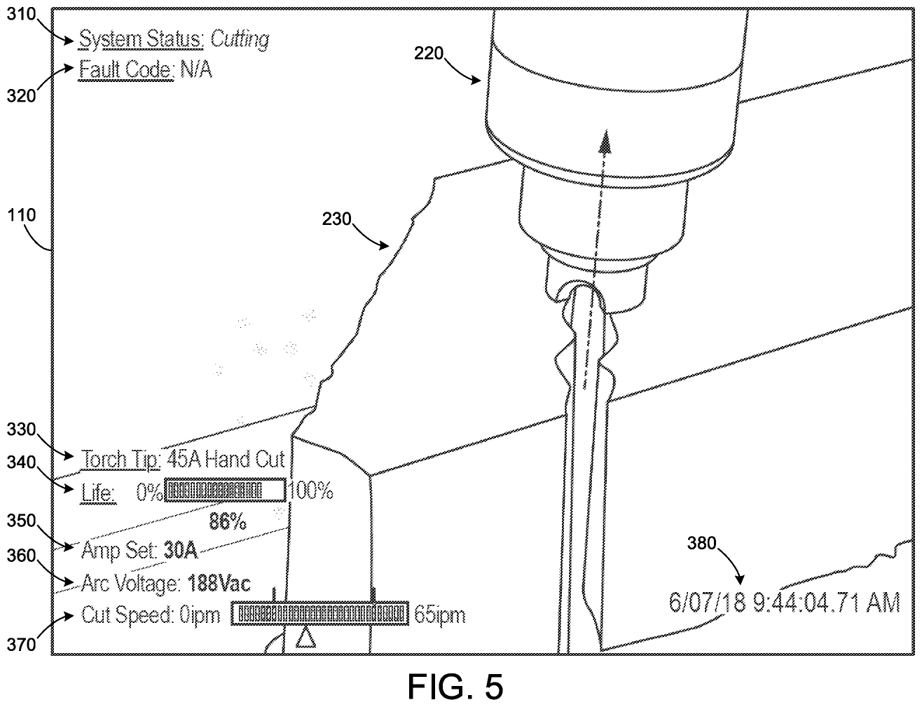

The system 200 can assist an operator in making precise cuts by directing them to cut within the desired cut path 390. In some embodiments, the torch 220 can automatically shut off if the operator begins cutting beyond the desired cut path 390. Referring to FIG. 4, the display 110 of the protective helmet 100 shows the initiation of the arc and start of the cutting operation. Here we can see that the cut path and torch angularity indicator 390 is still overlaid and visible, and the operator has aligned the torch 220 with the desired path to perform the operation. Referring to FIG. 5, the display 110 of the protective helmet 100 shows the torch system 210 during the cutting operation. As illustrated by cut speed indicator 370, the operator is moving at a cut speed within the optimal/desired range and is receiving positive feedback from the augmented reality system 200. The operator is also moving the torch 220 along the desired cut path 390.

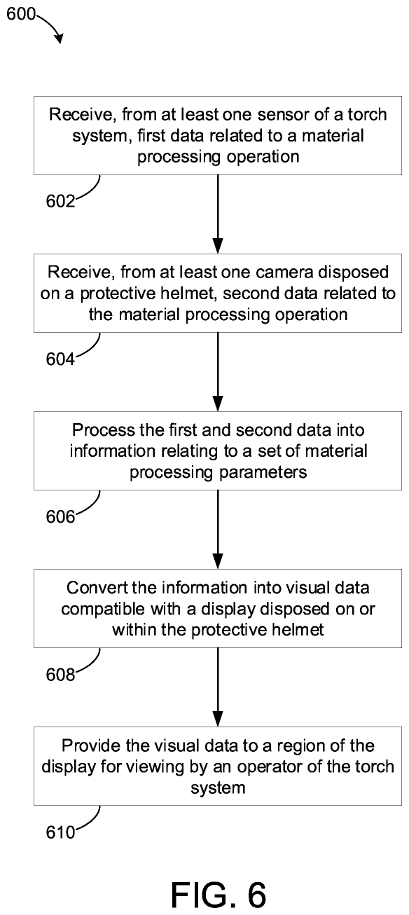

Referring to FIG. 6, a process 600 for visually communicating material processing parameters to an operator of a torch system 210 is illustrated. The process 600 begins by receiving, from at least one sensor 240 of a torch system 210, first data related to a material processing operation in step 602. For example, the sensor 240 can include at least one of an accelerometer or a gyroscope disposed on or within the torch 220. In some embodiments, the at least one sensor 240 is configured to monitor motion of the torch 220 during the material processing operation.

Process 600 continues by receiving, from at least one camera 120 disposed on a protective helmet 100, second data related to the material processing operation in step 604. For example, the camera 120 can capture images and/or video of the workpiece 230 and torch 220 to be processed by processor 140. Process 600 continues by processing the first and second data into information relating to a set of material processing parameters in step 606. For example, processor 140 can process the first data received from the at least one sensor 240 and second data received from camera 120 using memory 150. In some embodiments, processor 140 can process the first and second data to determine a velocity of the torch 220 with respect to the workpiece 230. In some embodiments, processor 140 can process the first and second data to determine an angle of the torch 220 with respect to the workpiece 230.

Process 600 continues by converting the information into visual data compatible with a display 110 disposed on or within the protective helmet 100 in step 608. For example, processor 140 can convert the velocity of the torch 220 with respect to the workpiece 230 into a numerical value that can be displayed using cut speed indicator 370 of display 110. In some embodiments, processor 140 can convert the angle of the torch 220 with respect to the workpiece 230 into a numerical value that can be displayed on display 110. Process 600 finishes by providing the visual data to a region of the display 110 for viewing by an operator of the torch system 210 in step 610. For example, the visual data can be displayed using system status indicator 310, fault code indicator 320, torch process type 330, torch tip life indicator 340, amperage setting indicator 350, arc voltage indicator 360, cut speed indicator 370, and date and time indicator 380. In some embodiments, the visual data can be transferred to a second display located at a distance from the protective helmet 100. The visual data can be provided to a second region of the second display for viewing by a second operator.

In some embodiments, a sensor 160 disposed on or within the protective helmet 100 can provide additional data related to the material processing operation. For example, system 200 can receive, from at least one temperature sensor 160 disposed on or within the protective helmet 100, third data related to the material processing operation. Processor 140 can process the third data into temperature information relating to a temperature of a region of the workpiece 230. Processor 140 can also convert the temperature information into second visual data compatible with the display 110. The second visual data can be provided to the region of the display 110 for viewing by the operator of the torch system 210. For example, the second visual data can be an alert indicating the temperature of the region of the workpiece 230.

In some embodiments, system 200 can receive, from a light spectrometer 160 disposed on or within the protective helmet 100, third data related to the material processing operation. Processor 140 can process the third data into wavelength information relating to a wavelength of a light emitted from the torch system 210. The processor 140 can also convert the wavelength information into second visual data compatible with the display 110. The second visual data can be provided to the region of the display 110 for viewing by the operator of the torch system 210.

In some embodiments, system 200 can receive, from a microphone 130 disposed on or within the protective helmet 100, audio data related to a command from the operator of the torch system 210. Processor 140 can process the visual data into adjusted visual data based on the command from the operator of the torch system 210. The visual data can be provided to the region of the display 110 for viewing by the operator of the torch system 210.

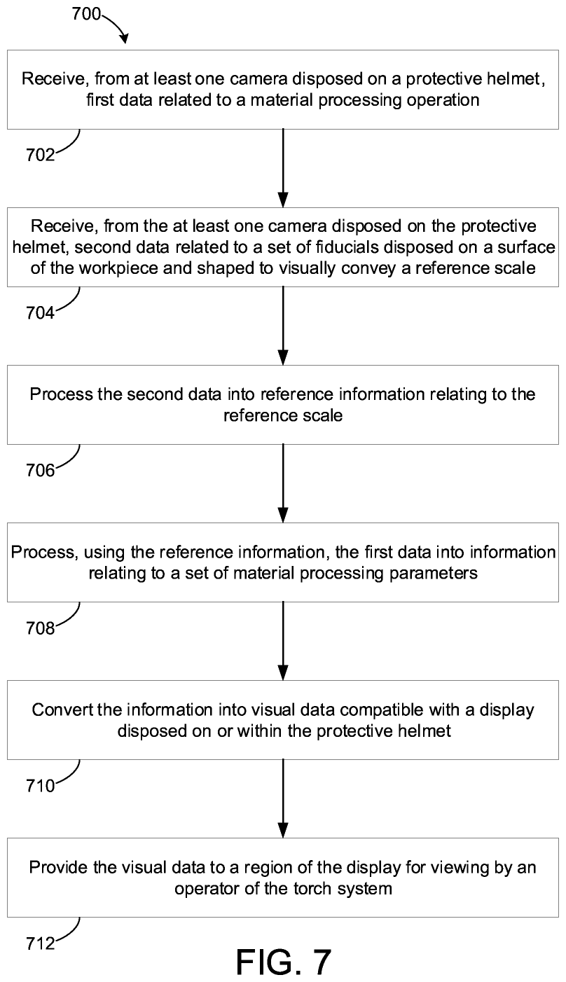

Referring to FIG. 7, a process 700 for visually communicating material processing parameters to an operator of a torch system 210 is illustrated. The process 700 begins by receiving, from at least one camera 120 disposed on a protective helmet 100, first data related to a material processing operation of a torch system 210 in step 702. For example, the camera 120 can capture images and/or video of the workpiece 230 and torch 220 to be processed by processor 140 to determine, for example, the movement of the torch 220 relative to the workpiece 230.

Process 700 continues by receiving, from the at least one camera 120 disposed on the protective helmet 100, second data related to a set of fiducials 250 disposed on a surface of the workpiece 230 in step 704. The set of fiducials 250 can be shaped to visually convey a reference scale. For example, the camera 120 can capture images and/or video of fiducials 250 to be processed by processor 140 to determine a reference scale. In some embodiments, the set of fiducials 250 are equally spaced apart. In some embodiments, the set of fiducials 250 include at least two anchor fiducials.

Process 700 continues by processing the second data into reference information relating to the reference scale in step 706. For example, processor 140 can process the second data to determine a distance between the set of fiducials and a reference scale based on the distance. Process 700 continues by processing, using the reference information, the first data into information relating to a set of material processing parameters in step 708. For example, processor 140 can process the first data received from the camera 120 using the reference scale. The reference scale allows processor 140 to determine accurate information regarding the movement of the torch 220 with respect to the workpiece 230. In some embodiments, processor 140 can process the first data using the reference scale to determine a velocity of the torch 220 with respect to the workpiece 230. In some embodiments, processor 140 can process the first data using the reference scale to determine an angle of the torch 220 with respect to the workpiece 230.

Process 700 continues by converting the information into visual data compatible with a display 110 disposed on or within the protective helmet 100 in step 710. For example, processor 140 can convert the velocity of the torch 220 with respect to the workpiece 230 into a numerical value that can be displayed using cut speed indicator 370 of display 110. In some embodiments, processor 140 can convert the angle of the torch 220 with respect to the workpiece 230 into a numerical and/or color-coded value that can be displayed on display 110. Process 700 finishes by providing the visual data to a region of the display 110 for viewing by an operator of the torch system 210 in step 712. For example, the visual data can be displayed using system status indicator 310, fault code indicator 320, torch process type 330, torch tip life indicator 340, amperage setting indicator 350, arc voltage indicator 360, cut speed indicator 370, and date and time indicator 380. The visual data being visible to the operator during processing to provide real time feedback of performance.

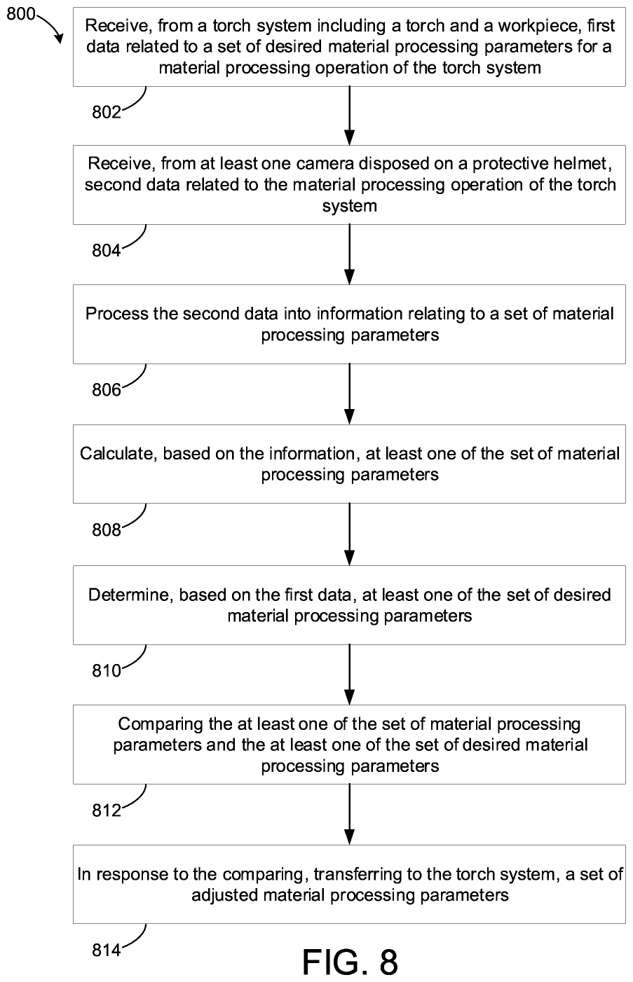

Referring to FIG. 8, a process 800 for controlling material processing parameters of a torch system 210 is illustrated. The process 800 begins by receiving, from a torch system 210 comprising a torch 220 and a workpiece 230, first data related to a set of desired material processing parameters for a material processing operation of a torch system 210 in step 802. For example, communication circuitry 170 of the protective helmet 100 can receive the first data from communication circuitry 270 of the torch system 210. In some embodiments, communication circuitry 170 can receive the first data from communication circuitry 270 using Bluetooth, Wi-Fi, or any comparable data transfer connection.

Process 800 continues by receiving, from at least one camera 120 disposed on a protective helmet 100, second data related to the material processing operation of the torch system 210 in step 804. For example, the camera 120 can capture images and/or video of the workpiece 230 and torch 220 to be processed by processor 140 to determine, for example, the movement of the torch 220 relative to the workpiece 230.

Process 800 continues by processing the second data into information relating to a set of material processing parameters in step 806. For example, processor 140 can process the second data received from camera 120 using memory 150. Process 800 continues by calculating, based on the information, at least one of the set of material processing parameters in step 808. For example, processor 140 can calculate a velocity of the torch 220 with respect to the workpiece 230 using the information. In some embodiments, processor 140 can calculate an angle of the torch 220 with respect to the workpiece 230 using the information. In some embodiments, processor 140 can calculate a length of the material processing operation using the information. For example, processor 140 can calculate how long of a cut has been performed using the second data received from camera 120. In some embodiments, processor 140 can calculate a distance between the torch 220 and an edge of the workpiece 230.

Process 800 continues by determining, based on the first data, at least one of the set of desired material processing parameters in step 810. In some embodiments, at least one of the set of desired material processing parameters includes a desired velocity of the torch 220 relative to the workpiece 230. In some embodiments, at least one of the set of desired material processing parameters includes a desired length of the material processing operation. In some embodiments, at least one of the set of desired material processing parameters includes a threshold distance between the torch 220 and the edge of the workpiece 230.

Process 800 continues by comparing the at least one of the set of material processing parameters and the at least one of the set of desired material processing parameters in step 812 and finishes by, in response to the comparing, transferring, to the torch system 210, a set of adjusted material processing parameters in step 814. For example, if the system 200 determines that the velocity of the torch 220 relative to the workpiece 230 is different than the desired velocity of the torch 220 relative to the workpiece 230, the system 200 transfers the set of adjusted material processing parameters to the torch system 210 using communication circuitry 170 and communication circuitry 270. In some embodiments, one of the set of adjusted material processing parameters includes an operating current of the torch 220. For example, if the system 200 determines that the velocity of the torch 220 relative to the workpiece 230 is different than the desired velocity of the torch 220 relative to the workpiece 230, processor 280 can adjust the operating current delivered by the power supply 260 to the torch 220 to compensate for the desired velocity variance (e.g., increased current if going faster than the desired velocity or decreased current if going slower than the desired velocity). In some embodiments, system 200 can detect/anticipate a kerf in the cut path and adjust the operating current delivered by the power supply 260 to assist the plasma arc and operator in navigating the kerf (e.g., increasing the current as the plasma arc arrives at the kerf and decreasing the current once the plasma arc bridges/crosses the kerf).

In some embodiments, if the system 200 determines that the length of the material processing operation is greater than or equal to the desired length of the material processing operation, the system 200 ceases the material processing operation of the torch system 210. For example, if the system 200 determines that desired cut length has been reached, the system 200 can terminate the cutting operation of the torch 220 to prevent a longer cut.

In some embodiments, if the system 200 determines that the distance between the torch 220 and the edge of the workpiece 230 is less than or equal to the threshold distance, the system 200 initiates a torch shutdown sequence of the torch system 210. For example, if the system 200 determines that the torch 220 is approaching the edge of the workpiece 230, the system 200 can initiate a torch shutdown sequence automatically in order to prevent damage to the torch 220.

The augmented reality system 200 is capable of providing a multitude of information to an operator to generate a desired outcome. In one embodiment, the augmented reality system 200 can process data/inputs to provide an indication of torch 220 angularity relative to the workpiece 230. In one embodiment, the augmented reality system 200 can process data/inputs to notify the shop or shop elements that a process is almost complete. In one embodiment, the augmented reality system 200 can process data/inputs to provide cut quality analysis and storage for future processes. In one embodiment, the augmented reality system 200 can process data/inputs to provide process monitoring which can watch for tip ups and adjust the nest and motion in real time/accordingly to move around tip ups or other defects or obstacles. In one embodiment, the augmented reality system 200 can process data/inputs to provide analysis of after the cut remnants, identifying, storing, and/or recalling this data to maximize material consumption without extensive serial numbers or identification. In some embodiments, camera 120 of augmented reality system 200 can monitor and/or certify parts cut from a workpiece (e.g., compare dimensions and tolerances to a CNC file) for quality assurance and certification. For example, alerting an operator that parts are out of code or close to the limits of the part tolerances and/or indicating trouble spots on a part being repetitively cut out by the operator, thereby allowing them to adjust their technique and create higher quality parts.

In one embodiment, the augmented reality system 200 can identify defects in the consumables (e.g., a ding in the bore of the nozzle or too large a dimple in an electrode). The augmented reality system 200 can include a service type application to help identify the location of a component in view of a particular error code. Tech service can obtain permission to see the operator's field of view to help with remote troubleshooting or remote training. Serial codes, part numbers can be displayed over the torch 220 and consumable, and can be ordered directly or tied to a customer's system for reorder requests. In one embodiment of the invention, part quality validation can be achieved. The camera 120 of the protective helmet 100 can inspect the part that has been cut relative to a CNC part file to validate that the part has been cut to within specifications. Ported cut features could be identified and the code that created the feature can also be presented with changes to that code.

In one embodiment, the augmented reality system 200 can process data/inputs to provide analysis of the cutting table that workpiece 230 is on. The analysis can provide information from the harmonics of table motion to identify damaged or about to fail rack, gear, cables, etc. In one embodiment, the augmented reality system can process data/inputs to provide operational playback by recording and also overlaying cut statistics, lines, and color codes. The operational playback can show where the cut speed might have been too fast or slow such that the operator can then attribute that to edge quality results. In one embodiment, the protective helmet 100 can include an ohmic contact input which can be used as a point selector. Using the ohmic contact input, when the torch 220 touches the workpiece 230 and closes the ohmic circuit, the system 210 can determine that a selection point input as been selected. Two points, for example, could be used to generate a line. Additionally, a constant contact between the torch 220 and the workpiece 230 can be used to draw with the torch 220.

In one embodiment, the augmented reality system 200 can process data/inputs to provide twin simulation analysis, for example after a cut is traced or planned, the system 200 can then play a digital twin simulation of the proposed direction and speed to achieve the best quality cut by hand. In one embodiment, the augmented reality system 200 can process data/inputs to provide a digital twin simulation for a robotic application or table application prior to actual execution to make sure there will be no crashes or obstructions. In one embodiment, the augmented reality system 200 can process data/inputs to provide system status, warning, notifications, and put them on a heads-up display for an operator who may be running multiple tables so they can minimize down time.

The systems and methods described herein provide a number of benefits over the current state of the art, the advantages including: operator can inspect workpiece 230 in between cuts without lifting the protective helmet 100; operator can detect fault codes using fault code indicator 320 without lifting the protective helmet 100; operator can determine the life of consumables before completing a cutting operation using consumable life indicator 340; inexperienced operators can be given feedback on cut speed and other training feedback; any information can be provided on the operator s field of view; operator can confirm the right components are installed more easily; operator can be aware of system status without the need to be near the power supply 260 using system status indicator 310; tech support can be given to an operator without lifting the protective helmet 100; operator can see workpiece 230 more clearly with augmented reality system 200 compared to the tint or dimness of traditional eye protection; operator can see workpiece 230 more clearly with augmented reality system 200 during processing operations and between processing operations.

The above-described techniques can be implemented in digital and/or analog electronic circuitry, or in computer hardware, firmware, software, or in combinations of them. The implementation can be as a computer program product, i.e., a computer program tangibly embodied in a machine-readable storage device, for execution by, or to control the operation of, a data processing apparatus, e.g., a programmable processor, a computer, and/or multiple computers. A computer program can be written in any form of computer or programming language, including source code, compiled code, interpreted code and/or machine code, and the computer program can be deployed in any form, including as a stand-alone program or as a subroutine, element, or other unit suitable for use in a computing environment. A computer program can be deployed to be executed on one programmable processor or on multiple programmable processors.

Processors 140 and 280 can perform the above-described method steps by executing a computer program to perform functions of the invention by operating on input data and/or generating output data. Method steps can also be performed by, and an apparatus can be implemented as, special purpose logic circuitry, e.g., a FPGA (field programmable gate array), a FPAA (field-programmable analog array), a CPLD (complex programmable logic device), a PSoC (Programmable System-on-Chip), ASH′ (application-specific instruction-set processor), or an ASIC (application-specific integrated circuit), or the like. Subroutines can refer to portions of the stored computer program and/or the processor, and/or the special circuitry that implement one or more functions.

Processors 140 and 280 may include, by way of example, special purpose microprocessors specifically programmed with instructions executable to perform the methods described herein, and any one or more processors of any kind of digital or analog computer. Generally, a processor receives instructions and data from a read-only memory or a random access memory or both. The essential elements of a computer are a processor for executing instructions and one or more memory devices for storing instructions and/or data. Memory devices 150 and 290 can be used to temporarily store data, such as a cache. Memory devices 150 and 290 can also be used for long-term data storage. Computer-readable storage mediums suitable for embodying computer program instructions and data include all forms of volatile and non-volatile memory, including by way of example semiconductor memory devices, e.g., DRAM, SRAM, EPROM, EEPROM, and flash memory devices; magnetic disks, e.g., internal hard disks or removable disks; magneto-optical disks; and optical disks, e.g., CD, DVD, HD-DVD, and Blu-ray disks. The processor and the memory can be supplemented by and/or incorporated in special purpose logic circuitry.

Display 110 can be a display device, e.g., a CRT (cathode ray tube), plasma, or LCD (liquid crystal display) monitor, a mobile computing device display or screen, a holographic device and/or projector, for displaying information to the operator. The operator can use a keyboard and/or a pointing device, e.g., a mouse, a trackball, a touchpad, or a motion sensor to provide input to the augmented reality system 200 (e.g., interact with a user interface element). Other kinds of devices can be used to provide for interaction with an operator as well; for example, feedback provided to the operator can be any form of sensory feedback, e.g., visual feedback, auditory feedback, or tactile feedback; and input from the operator can be received in any form, including acoustic, speech, and/or tactile input.

The components of the augmented reality system 200 can be interconnected by communication circuitry 170 and 270 using transmission medium, which can include any form or medium of digital or analog data communication (e.g., a communication network). Transmission medium can include one or more packet-based networks and/or one or more circuit-based networks in any configuration. Packet-based networks can include, for example, the Internet, a carrier internet protocol (IP) network (e.g., local area network (LAN), wide area network (WAN), campus area network (CAN), metropolitan area network (MAN), home area network (HAN)), a private IP network, an IP private branch exchange (IPBX), a wireless network (e.g., radio access network (RAN), Bluetooth, near field communications (NFC) network, Wi-Fi, WiMAX, general packet radio service (GPRS) network, HiperLAN), and/or other packet-based networks. Circuit-based networks can include, for example, the public switched telephone network (PSTN), a legacy private branch exchange (PBX), a wireless network (e.g., RAN, code-division multiple access (CDMA) network, time division multiple access (TDMA) network, global system for mobile communications (GSM) network), and/or other circuit-based networks.

Communication circuitry 170 and 270 can use one or more communication protocols to transfer information over transmission medium. Communication protocols can include, for example, Ethernet protocol, Internet Protocol (IP), Voice over IP (VOiP), a Peer-to-Peer (P2P) protocol, Hypertext Transfer Protocol (HTTP), Session Initiation Protocol (SIP), H.323, Media Gateway Control Protocol (MGCP), Signaling System #7 (SS7), a Global System for Mobile Communications (GSM) protocol, a Push-to-Talk (PTT) protocol, a PTT over Cellular (POC) protocol, Universal Mobile Telecommunications System (UMTS), 3GPP Long Term Evolution (LTE) and/or other communication protocols.

One skilled in the art will realize the invention can be embodied in other specific forms without departing from the spirit or essential characteristics thereof. The foregoing embodiments are therefore to be considered in all respects illustrative rather than limiting of the invention described herein. It will be appreciated that the illustrated embodiments and those otherwise discussed herein are merely examples of the invention and that other embodiments, incorporating changes thereto, including combinations of the illustrated embodiments, fall within the scope of the invention.

What is claimed:

1. A method for visually communicating material processing parameters to an operator of a torch system, the method comprising:
receiving, from at least one sensor of a torch system, first data related to a material processing operation;
receiving, from at least one camera disposed on a protective helmet, second data related to the material processing operation;
processing the first and second data into information relating to a set of material processing parameters;
converting the information into visual data compatible with a display disposed on or within the protective helmet; and
providing the visual data to a region of the display for viewing by an operator of the torch system, wherein the region is within a field of view of the operator.

2. The method of claim 1, wherein the torch system comprises a torch and a workpiece.

3. The method of claim 2, wherein the at least one sensor is disposed on or within the torch.

4. The method of claim 3, wherein the at least one sensor comprises at least one of an accelerometer or a gyroscope.

5. The method of claim 4, wherein the at least one sensor is configured to monitor motion of the torch during the material processing operation.

6. The method of claim 2, wherein the set of material processing parameters comprises at least one of a velocity of the torch with respect to the workpiece and an angle of the torch with respect to the workpiece.

7. The method of claim 1, further comprising:
receiving, from at least one temperature sensor disposed on or within the protective helmet, third data related to the material processing operation;
processing the third data into temperature information relating to a temperature of a region of the workpiece;
converting the temperature information into second visual data compatible with the display; and
providing the second visual data to the region of the display for viewing by the operator of the torch system.

8. The method of claim 7, wherein the second visual data comprises an alert indicating the temperature of the region of the workpiece.

9. The method of claim 1, further comprising:
receiving, from a light spectrometer disposed on or within the protective helmet, third data related to the material processing operation;
processing the third data into wavelength information relating to a wavelength of a light emitted from the torch system;
converting the wavelength information into second visual data compatible with the display; and
providing the second visual data to the region of the display for viewing by the operator of the torch system.

10. The method of claim 1, further comprising:
receiving, from a microphone disposed on or within the protective helmet, audio data related to a command from the operator of the torch system;
processing the visual data into adjusted visual data based on the command from the operator of the torch system; and
providing the visual data to the region of the display for viewing by the operator of the torch system.

11. The method of claim 1, further comprising:
transferring the visual data to a second display located at a distance from the protective helmet; and
providing the visual data to a second region of the second display for viewing by a second operator.

12. A method for visually communicating material processing parameters to an operator of a torch system, the method comprising:
receiving, from at least one camera disposed on a protective helmet, first data related to a material processing operation of a torch system, wherein the torch system comprises a torch and a workpiece;
receiving, from the at least one camera disposed on the protective helmet, second data related to a set of fiducials disposed on a surface of the workpiece, wherein the set of fiducials are shaped to visually convey a reference scale;
processing the second data into reference information relating to the reference scale;
processing, using the reference information, the first data into information relating to a set of material processing parameters;
converting the information into visual data compatible with a display disposed on or within the protective helmet; and
providing the visual data to a region of the display for viewing by an operator of the torch system, wherein the region is within a field of view of the operator.

13. The method of claim 12, wherein the set of fiducials are equally spaced apart.

14. The method of claim 12, wherein the set of fiducials comprises at least two anchor fiducials.

15. The method of claim 12, wherein the set of material processing parameters comprises at least one of a velocity of the torch with respect to the workpiece and an angle of the torch with respect to the workpiece.

16. The method of claim 12, further comprising:
receiving, from at least one temperature sensor disposed on or within the protective helmet, third data related to the material processing operation;
processing the third data into temperature information relating to a temperature of a region of the workpiece;
converting the temperature information into second visual data compatible with the display; and
providing the second visual data to the region of the display for viewing by the operator of the torch system.

17. The method of claim 16, wherein the second visual data comprises an alert indicating the temperature of the region of the workpiece.

18. The method of claim 12, further comprising:
receiving, from a light spectrometer disposed on or within the protective helmet, third data related to the material processing operation;
processing the third data into wavelength information relating to a wavelength of a light emitted from the torch system;
converting the wavelength information into second visual data compatible with the display; and
providing the second visual data to the region of the display for viewing by the operator of the torch system.

19. The method of claim 12, further comprising:
receiving, from a microphone disposed on or within the protective helmet, audio data related to a command from the operator of the torch system;
processing the visual data into adjusted visual data based on the command from the operator of the torch system; and
providing the visual data to the region of the display for viewing by the operator of the torch system.

20. The method of claim 12, further comprising:
transferring the visual data to a second display located at a distance from the protective helmet; and
providing the visual data to a second region of the second display for viewing by a second operator.

21. A method for controlling material processing parameters of a torch system, the method comprising:
receiving, from a torch system comprising a torch and a workpiece, first data related to a set of desired material processing parameters for a material processing operation of the torch system;
receiving, from at least one camera disposed on a protective helmet, second data related to the material processing operation of the torch system;
processing the second data into information relating to a set of material processing parameters;
calculating, based on the information, at least one of the set of material processing parameters;
determining, based on the first data, at least one of the set of desired material processing parameters;
comparing the at least one of the set of material processing parameters and the at least one of the set of desired material processing parameters; and
in response to the comparing, transferring, to the torch system, a set of adjusted material processing parameters.

22. The method of claim 21, wherein the at least one of the set of material processing parameters comprises a velocity of the torch relative to the workpiece and the at least one of the set of desired material processing parameters comprises a desired velocity of the torch relative to the workpiece.

23. The method of claim 22, wherein determining that the velocity of the torch is different than the desired velocity of the torch results in the transferring of the set of adjusted material processing parameters.

24. The method of claim 23, wherein one of the set of adjusted material processing parameters comprises an operating current of the torch.

25. The method of claim 22, wherein the at least one of the set of material processing parameters comprises a length of the material processing operation and the at least one of the set of desired material processing parameters comprises a desired length of the material processing operation.

26. The method of claim 25, wherein determining that the length is greater than or equal to the desired length results in the transferring of the set of adjusted material processing parameters.

27. The method of claim 26, further comprising ceasing the material processing operation of the torch system.

28. The method of claim 22, wherein the at least one of the set of material processing parameters comprises a distance between the torch and an edge of the workpiece and the at least one of the set of desired material processing parameters comprises a threshold distance between the torch and the edge of the workpiece.

29. The method of claim 28, wherein determining that the distance between the torch and the edge of the workpiece is less than or equal to the threshold distance results in the transferring of the set of adjusted material processing parameters.

30. The method of claim 29, further comprising initiating a torch shutdown sequence at the torch system.
